(19) United States
(12) Patent Application Publication
(10) Pub. No.: US 2018/0117013 A1
(43) Pub. Date: May 3, 2018

(54) METHODS OF TREATING LIVER DISEASE USING INDANE ACETIC ACID DERIVATIVES

(71) Applicant: T3D THERAPEUTICS, INC., Research Triangle Park, NC (US)

(72) Inventor: John Robert DIDSBURY, Raleigh, NC (US)

(73) Assignee: T3D Therapeutics, Inc., Research Triangle Park, NC (US)

(21) Appl. No.: 15/561,434

(22) PCT Filed: Mar. 23, 2016
(86) PCT No.: PCT/US2016/023694

§ 371 Date: Sep. 25, 2017

Related U.S. Application Data
(60) Provisional application No. 62/138,698, filed on Mar. 26, 2015.

(51) Int. Cl. A61K 31/421; A61K 31/426; A61K 45/06; A61P 1/16

(52) U.S. Cl. CPC A61K 31/421; A61P 1/16; A61K 45/06; A61K 31/426

(57) ABSTRACT

This invention describes the use of indane acetic acid derivatives which are dual PPAR delta/gamma agonists for the treatment of liver diseases including one or more of the following: NAFLD (Non Alcoholic Fatty Liver Disease), NASH (Non Alcoholic Steatohepatitis), Farber's Disease, ACLF (Acute-on-Chronic Liver Failure), CLF (Chronic Liver Failure), POLT-HCV-SVR (Post-Orthotopic Liver Transplant due to Hepatitis C Virus infection after Sustained Viral Response following anti-HCV therapy), Alagille syndrome, PFIC (Progressive Familial Intrahepatic Cholestasis), PBC (Primary Biliary Cirrhosis), Primary Sclerosing Cholangitis, ADPCLD (Autosomal Dominant Polycystic Liver Disease), Treatment of liver transplant patients with reestablished fibrosis, CESD (Cholesteryl Ester Storage Disease), SHTG (Severe Hypertriglyceridemia), HoFH (Homozygous Familial Hypercholesterolemia), HE (Hepatic Encephalopathy), or Alcoholic Liver Disease.

This application claims priority of U.S. provisional application No. 62/138,698 filed on Mar. 26, 2015, which is incorporated in its entirety by reference.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

### A. Field of the Invention

The present invention relates to the use of indane acetic acids and their derivatives, which are dual PPAR delta and gamma agonists, for the treatment of NAFLD (Non Alcoholic Fatty Liver Disease), NASH (Non Alcoholic Steatohepatitis), Farber's Disease, ACLF (Acute-on-Chronic Liver Failure), CLF (Chronic Liver Failure), POLT-HCV-SVR (Post-Orthotopic Liver Transplant due to Hepatitis C Virus infection after Sustained Viral Response following anti-HCV therapy), Alagille syndrome, PFIC (Progressive Familial Intrahepatic Cholestasis), PBC (Primary Biliary Cirrhosis), Primary Sclerosing Cholangitis, ADPCLD (Autosomal Dominant Polycystic Liver Disease), treatment of liver transplant patients with reestablished fibrosis, CESD (Cholesteryl Ester Storage Disease), SHTG (Severe Hypertriglyceridemia), HoFH (Homozygous Familial Hypercholesterolemia), HE (Hepatic Encephalopathy), or Alcoholic Liver Disease, not previously treated by such activity.

### B. Description of Related Art

The art is well aware of treatment of various diseases with compositions which have dual peroxisome proliferator activated receptor (PPAR) alpha and gamma agonist activity as well as each of alpha, gamma and delta agonists individually. Only recently have there been discovered compositions with dual PPAR delta and gamma agonist activity where delta activity is greater than gamma activity, and gamma activity is greater than alpha activity. Little is known about any benefits to their use beyond what's known about the other more well-known activities. In addition, there appears to be one composition with dual PPAR alpha and delta activity. There are a number of Orphan diseases that are not currently well serviced and, frequently, little research is done into these diseases' states. One such disease is Primary Biliary Cirrhosis (PBC).

Nonalcoholic Steatohepatitis (NASH) is a common, often “silent”, liver disease. It resembles alcoholic liver disease, but occurs in people who drink little or no alcohol. The major feature in NASH is fat in the liver, along with inflammation and damage. Most people with NASH feel well and are not aware that they have a liver problem. Nevertheless, NASH can be severe and can lead to cirrhosis of the liver, in which the liver is permanently damaged and scarred and no longer able to function properly.

Nonalcoholic Fatty Liver Disease (NAFLD) is a fatty liver disease common in chronic liver disease subjects. Excess liver fat can lead to liver complications. While not alcohol-related, these conditions can be related to obesity, diet, and other health-related issues.

Individuals with elevated liver enzymes and/or one having a fatty liver (e.g. determined by ultrasound or fatty liver index) are considered to have NASH or NAFLD. A reduction in enzymes, fat, or fatty liver index is an indicator of an improving or corrected condition.

This and other diseases are still in search of adequate treatments.

### SUMMARY OF THE INVENTION

The present invention provides methods of treating and/or preventing the following liver diseases:
1. NAFLD
2. NASH
3. Farber's Disease
4. ACLF (acute-on-chronic liver failure)
5. CLF (chronic liver failure)
6. POLT-HCV-SVR post-orthotopic liver transplant, or POLT, due to hepatitis C virus, or HCV, infection and have subsequently achieved sustained viral response, or SVR, following anti-HCV therapy
7. Alagille syndrome
8. Progressive Familial Intrahepatic cholestasis (PFIC)
9. Primary Biliary Cirrhosis (PBC)
10. Primary Sclerosing Cholangitis
11. Autosomal Dominant Polycystic Liver Disease (ADPCLD)
12. Treatment of liver transplant patients with reestablished fibrosis
13. Cholesteryl Ester Storage Disease (CESD)
14. Severe Hypertriglyceridemia (SHTG)
15. Homozygous Familial Hypercholesterolemia (HoFH)
16. Hepatic Encephalopathy (HE)
17. Alcoholic Liver Disease

The methods include administering to a subject in need thereof an effective amount of a PPAR delta and gamma dual agonist such as a compound of Formula I:

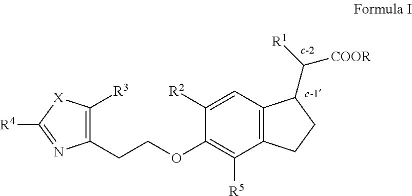

wherein in Formula I

R is H or C₁-C₆ alkyl;

R¹ is H, COOR, C₃-C₈ cycloalkyl, or C₁-C₆ alkyl, C₂-C₆ alkenyl, or C₁-C₆ alkoxy each of which may be unsubstituted or substituted with fluoro, methylenedioxyphenyl, or phenyl which may be unsubstituted or substituted with R⁶;

R² is H, halo, or C₁-C₆ alkyl which may be unsubstituted or substituted with C₁-C₆ alkoxy, oxo, fluoro, or

R² is phenyl, furyl, thienyl, pyrrolyl, oxazolyl, thiazolyl, imidazolyl, pyrazolyl, isoxazolyl, isothiazolyl, triazolyl, oxadiazolyl, thiadiazolyl, tetrazolyl, pyridyl, pyrrolidinyl, piperidinyl, tetrahydropyranyl, tetrahydrothiopyranyl, piperazinyl, or morpholinyl,
each of which may be unsubstituted or substituted with R⁶;

R³ is H, C₁-C₆ alkyl, or phenyl, which may be unsubstituted or substituted with R⁶;

X is O or S;

R⁴ is phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benxothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1,4-benzodioxanyl,

each of which may be unsubstituted or singularly or multiply substituted with R⁶, or with phenyl, furyl, thienyl, pyrrolyl, oxazolyl, thiazolyl, imidazolyl, pyrazolyl, isoxazolyl, isothiazolyl, triazolyl, oxadiazolyl, thiadiazolyl, tetrazolyl, pyridyl, pyrrolidinyl, piperidinyl, tetrahydropyranyl, tetrahydrothiopyranyl, piperazinyl, morpholinyl, benzodioxolyl, dihydrobenzofuranyl, indolyl, pyrimidinyl or phenoxy,

each of which may be unsubstituted or singularly or multiply substituted with R⁶;

R⁴ is C₁-C₆ alkyl or C₃-C₈ cycloalkyl, either of which may be unsubstituted or substituted with fluoro, oxo, or C₁-C₆ alkoxy which may be unsubstituted or substituted with C₁-C₆ alkoxy, or phenyl optionally substituted with R⁶,
each of which may be substituted with phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benzothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1,4-benzodioxanyl,
   each of which may be unsubstituted or further substituted with R⁶, or
C₁-C₆ alkyl may also be substituted with C₃-C₈ cycloalkyl or with phenoxy which may be unsubstituted or substituted with R⁶ or with phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benxothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1,4-benzodioxanyl,
   each of which may be unsubstituted or substituted with R⁶, or

R⁵ is H, halo or C₁-C₆ alkyl optionally substituted with oxo; and

R⁶ is halo, CF₃, C₁-C₆ alkyl optionally substituted with oxo or hydroxy, or C₁-C₆ alkoxy optionally substituted with fluoro; or a pharmaceutically acceptable salt, ester prodrug, stereoisomer, diastereomer, enantiomer, racemate or a combination thereof.

R³ may be attached to the heterocyclic moiety of the compound of Formula I at either the 4 or 5 position (i.e., at either available carbon atom) and, accordingly, the remaining portion of the molecule will be attached at the remaining available carbon atom.

In some embodiments, the compound of Formula I has structure as described above and R is potassium, sodium, calcium, magnesium, lysine, choline or meglumine salt thereof.

In other embodiments, for the compound of Formula I, R is H, R¹ is H, R² is H, R⁵ is H, R³ is C₁-C₆ alkyl, X is O, and R⁴ is a phenyl, singularly or multiply substituted with R⁶, wherein R⁶ is halo, CF₃, C₁-C₆ alkoxyl or C₁-C₆ alkyl, or a pharmaceutically acceptable salt thereof.

In other embodiments, for the compound of Formula I, R is H, R¹ is H, R² is H, R⁵ is H, R³ is C₁-C₆ alkyl, X is O, and R⁴ is a phenyl, singularly or multiply substituted with R⁶, wherein R⁶ is halo, CF₃, C₁-C₆ alkoxyl or C₁-C₆ alkyl, and the stereochemistry at C-1′ is defined as S, or a pharmaceutically acceptable salt thereof.

In other embodiments, for the compound of Formula I, R is H, R¹ is H, R² is H, R⁵ is H, R³ is C₁-C₆ alkyl, X is S, and R⁴ is a phenyl, singularly or multiply substituted with R⁶, wherein R⁶ is halo, CF₃, C₁-C₆ alkoxyl or C₁-C₆ alkyl, and the stereochemistry at C-1′ is defined as S, or a pharmaceutically acceptable salt thereof.

In other embodiments, for the compound of Formula I, R is H, R¹ is H, R² is F, R⁵ is H, R³ is C₁-C₆ alkyl, X is O, and R⁴ is a phenyl, singularly or multiply substituted with R⁶, wherein R⁶ is halo, CF₃, C₁-C₆ alkoxyl or C₁-C₆ alkyl, and the stereochemistry at C-1′ is defined as S, or a pharmaceutically acceptable salt thereof.

In other embodiments, for the compound of Formula I, R is H, R¹ is H, R² is H, R⁵ is F, or R² and R⁵ are F, R³ is C₁-C₆ alkyl, X is O, and R⁴ is a phenyl, singularly or multiply substituted with R⁶, wherein R⁶ is halo, CF₃, C₁-C₆ alkoxyl or C₁-C₆ alkyl, and the stereochemistry at C-1′ is defined as S, or a pharmaceutically acceptable salt thereof.

In other embodiments, for the compound of Formula I, R is H, R¹ is H, R² is H, R⁵ is H, R³ is C₁-C₆ alkyl, X is O, and R⁴ is a phenyl, singularly or multiply substituted with R⁶, wherein R⁶ is halo, CF₃, C₁-C₆ alkoxyl or C₁-C₆ alkyl, and the stereochemistry at C-1′ is defined as R, or a pharmaceutically acceptable salt thereof.

In one embodiment, the compound of Formula I is either the free acid or the potassium, sodium, calcium, magnesium, lysine, choline or meglumine salt of one of the following structures:

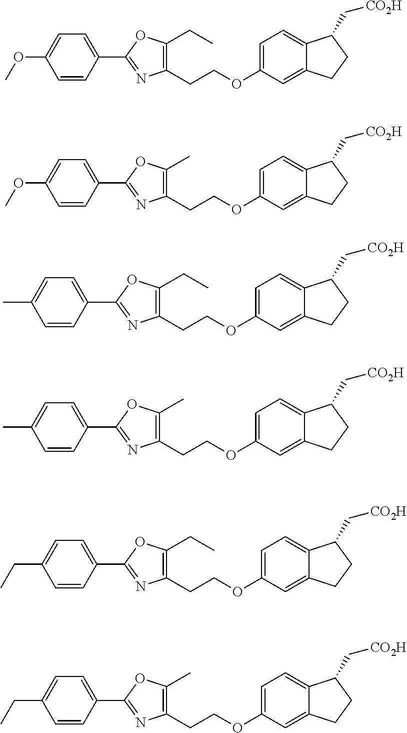 [SMILES: CCc1oc(-c2ccc(OC)cc2)nc1CCOc1ccc2c(c1)CC[C@H]2CC(=O)O ; COc1ccc(-c2nc(CCOc3ccc4c(c3)CC[C@H]4CC(=O)O)c(C)o2)cc1 ; CCc1oc(-c2ccc(C)cc2)nc1CCOc1ccc2c(c1)CC[C@H]2CC(=O)O ; Cc1ccc(-c2nc(CCOc3ccc4c(c3)CC[C@H]4CC(=O)O)c(C)o2)cc1 ; CCc1ccc(-c2nc(CCOc3ccc4c(c3)CC[C@H]4CC(=O)O)c(CC)o2)cc1 ; CCc1ccc(-c2nc(CCOc3ccc4c(c3)CC[C@H]4CC(=O)O)c(C)o2)cc1]

In another embodiment, the methods described herein may further include administration of one or more additional therapeutic agent.

Objects of the present invention will be appreciated by those of ordinary skill in the art from a reading of the referenced patent literature and the Examples and the detailed description of the embodiments, which follow, such description being merely illustrative of the present invention.

### DETAILED DESCRIPTION OF THE INVENTION

While this invention is susceptible to embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

### A. DEFINITIONS

The terms “about” and “essentially” mean±20 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended.

References throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are for the purpose of illustrating certain convenient embodiments of the present invention, and are not to be considered as limitation thereto. The term “means” preceding a present participle of an operation indicates a desired function for which there is one or more embodiments, i.e., one or more methods, devices, or apparatuses for achieving the desired function and that one skilled in the art could select from these, or their equivalent, in view of the disclosure herein and use of the term “means” is not intended to be limiting.

Generally, the nomenclature used herein and the laboratory procedures in organic chemistry, medicinal chemistry, and pharmacology described herein are those well known and commonly employed in the art. Unless defined otherwise, all technical and scientific terms used herein generally have the same meaning as commonly understood by one of ordinary skill in the art to which this disclosure belongs. In the event that there is a plurality of definitions for a term used herein, those in this section prevail unless stated otherwise.

As used herein the term “PPAR delta and gamma agonist” and “PPAR delta and gamma activity” refers to agonists where delta activity is greater than gamma activity and gamma activity is greater than alpha activity.

The term “halo” means F, Cl, Br, or I.

The term “C1-C6 alkyl” means a straight or branched saturated hydrocarbon carbon chain of from 1 to about 6 carbon atoms, respectively. Examples of such groups include methyl, ethyl, isopropyl, sec-butyl, 2-methylpentyl, n-hexyl, and the like.

The term “C2-C6 alkenyl” means a straight or branched unsaturated hydrocarbon carbon chain of from 2 to about 6 carbon atoms. Examples of such groups include vinyl, allyl, isopropenyl, 2-butenyl, 3-ethyl-2-butenyl, 4-hexenyl, and the like.

The term “C1-C6 haloalkyl” means a C1-C6 alkyl group substituted by 1 to 3 halogen atoms or fluorine up to the perfluoro level. Examples of such groups include trifluoromethyl, tetrafluoroethyl, 1,2-dichloropropyl, 6-iodohexyl, and the like.

The terms “C3-C6 cycloalkyl” and “C3-C8 cycloalkyl” mean a saturated carbocyclic ring system of from 3 to about 6 carbon atoms or from 3 to about 8 carbon atoms, respectively. Examples of such groups include cyclopropyl, cyclobutyl, cyclopentyl, cyclohexyl, and the like.

The term “C1-C6 acyl” means a C1-C6 alkyl group attached at the carbonyl carbon atom. The radical is attached to the rest of the molecule at the carbonyl bearing carbon atom. Examples of such groups include acetyl, propionyl, n-butanoyl, 2-methylpentantoyl, and the like.

The term “C1-C6 alkoxy” means a linear or branched saturated carbon group having from 1 to about 6 C atoms, said carbon group being attached to an O atom. The O atom is the point of attachment of the alkoxy substituent to the rest of the molecule. Such groups include, but are not limited to, methoxy, ethoxy, n-propoxy, isopropoxy, and the like.

The term “C1-C6 thioalkyl” means a linear or branched saturated carbon group having from 1 to about 6 C atoms, said carbon group being attached to an S atom. The S atom is the point of attachment of the thioalkyl substituent to the rest of the molecule. Such groups include, for example, methylthio, propylthio, hexylthio, and the like.

The term “C1-C6 haloalkoxy” means a C1-C6 alkoxy group further substituted on C with 1 to 3 halogen atoms or fluorine up to the perfluoro level.

The term “C3-C8 cycloalkoxy” means a C3-C8 cycloalkyl group attached to an O atom. The O atom is the point of attachment of the cycloalkoxy group with the rest of the molecule.

The term “phenoxy” means a phenyl group attached to an O atom. The O atom is the point of attachment of the phenoxy group to the rest of the molecule.

The term “6-membered heteroaryl ring” means a 6-membered monocyclic heteroaromatic ring radical containing 1-5 carbon atoms and up to the indicated number of N atoms. Examples of 6-membered heteroaryl rings are pyridyl, pyrimidyl, pyridazinyl, pyrazinyl, triazinyl, and the like.

The term “5- or 6-membered heterocyclic ring” means a 5- or 6-membered ring containing 1-5 C atoms and up to the indicated number of N, O, and S atoms, and may be aromatic, partially saturated, or fully saturated.

The term “optionally substituted” means that, unless indicated otherwise, the moiety so modified may have from one to up to the number of the substituents indicated, provided the resulting substitution is chemically feasible as recognized in the art. Each substituent may replace any H atom on the moiety so modified as long as the replacement is chemically possible and chemically stable. For example, a chemically unstable compound would be one where each of two substituents is bonded to a single C atom through each substituents heteroatom. Another example of a chemically unstable compound would be one where an alkoxy group is bonded to the unsaturated carbon of an alkene to form an enol ether. When there are two or more substituents on any moiety, each substituent is chosen independently of the other substituent so that, accordingly, the substituents can be the same or different.

When the 5- or 6-membered heterocyclic ring is attached to the rest of the molecule as a substituent, it becomes a radical. Examples of 5- or 6-membered heteroaryl ring radicals are furyl, pyrrolyl, thienyl, pyrazolyl, isoxazolyl, imidazolyl, oxazolyl, thiazolyl, isothiazolyl, triazolyl, thiadiazolyl, oxadiazolyl, pyridyl, pyrimidyl, pyridazinyl, pyrazinyl, triazinyl, and the like. Examples of partially unsaturated 5- or 6-membered heterocyclic ring radicals include dihydropyrano, pyrrolinyl, pyrazolinyl, imidazolinyl, dihydrofuryl, and the like. Examples of saturated 5- or 6-membered heterocyclic ring radicals include pyrrolidinyl, tetrahydropyridyl, piperidinyl, morpholinyl, tetrahydrofuryl, tetrahydrothienyl, piperazinyl, and the like. The point of attachment of the radical may be from any available C or N atom of the ring to the rest of the molecule. When the 5- or 6-membered heterocyclic ring is fused to another ring contained in the rest of the molecule, it forms a bicyclic ring. Examples of such 5- and 6-heterocyclic fused rings include pyrrolo, furo, pyrido, piperido, thieno, and the like. The point of fusion is at any available face of the heterocyclic ring and parent molecule.

The term “subject”, as used herein, means a mammalian subject (e.g., dog, cat, horse, cow, sheep, goat, monkey, etc.), and particularly human subjects (including both male and female subjects, and including neonatal, infant, juvenile, adolescent, adult and geriatric subjects, and further including various races and ethnicities including, but not limited to, white, black, Asian, American Indian and Hispanic).

As used herein, “treatment”, “treat”, and “treating” refer to reversing, alleviating, mitigating, or slowing the progression of, or inhibiting the progress of, a disorder or disease as described herein.

As used herein, “prevention”, “prevent”, and “preventing” refer to eliminating or reducing the incidence or onset of a disorder or disease as described herein, as compared to that which would occur in the absence of the measures taken.

As used herein, “an effective amount” refers to an amount that causes relief of symptoms of a disorder or disease as noted through clinical testing and evaluation, patient observation, and/or the like. An “effective amount” can further designate a dose that causes a detectable change in biological or chemical activity. The detectable changes may be detected and/or further quantified by one skilled in the art for the relevant mechanism or process. Moreover, an “effective amount” can designate an amount that maintains a desired physiological state, i.e., reduces or prevents significant decline and/or promotes improvement in the condition of interest. An “effective amount” can further refer to a therapeutically effective amount.

All patents, patent applications and publications referred to herein are incorporated by reference in their entirety. In case of a conflict in terminology, the present specification is controlling.

### B. COMPOUNDS

### (1). Formula I

The present invention encompasses the compounds of Formula I which are PPAR delta and gamma dual agonists,

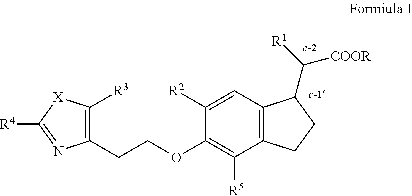

wherein in Formula I

R is H or C₁-C₆ alkyl;

R¹ is H, COOR, C₃-C₈ cycloalkyl, or C₁-C₆ alkyl, C₂-C₆ alkenyl, or C₁-C₆ alkoxy each of which may be unsubstituted or substituted with fluoro, methylenedioxyphenyl, or phenyl which may be unsubstituted or substituted with R⁶;

R² is H, halo, or C₁-C₆ alkyl which may be unsubstituted or substituted with C₁-C₆ alkoxy, oxo, fluoro, or

R² is phenyl, furyl, thienyl, pyrrolyl, oxazolyl, thiazolyl, imidazolyl, pyrazolyl, isoxazolyl, isothiazolyl, triazolyl, oxadiazolyl, thiadiazolyl, tetrazolyl, pyridyl, pyrrolidinyl, piperidinyl, tetrahydropyranyl, tetrahydrothiopyranyl, piperazinyl, or morpholinyl,
each of which may be unsubstituted or substituted with R⁶;

R³ is H, C₁-C₆ alkyl, or phenyl, which may be unsubstituted or substituted with R⁶;

X is O or S;

R⁴ is phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benxothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1,4-benzodioxanyl,

each of which may be unsubstituted or singularly or multiply substituted with R⁶, or with phenyl, furyl, thienyl, pyrrolyl, oxazolyl, thiazolyl, imidazolyl, pyrazolyl, isoxazolyl, isothiazolyl, triazolyl, oxadiazolyl, thiadiazolyl, tetrazolyl, pyridyl, pyrrolidinyl, piperidinyl, tetrahydropyranyl, tetrahydrothiopyranyl, piperazinyl, morpholinyl, benzodioxolyl, dihydrobenzofuranyl, indolyl, pyrimidinyl or phenoxy,

each of which may be unsubstituted or singularly or multiply substituted with R⁶;

R⁴ is C₁-C₆ alkyl or C₃-C₈ cycloalkyl, either of which may be unsubstituted or substituted with fluoro, oxo, or C₁-C₆ alkoxy which may be unsubstituted or substituted with C₁-C₆ alkoxy, or phenyl optionally substituted with R⁶,
each of which may be substituted with phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benzothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1,4-benzodioxanyl,
   each of which may be unsubstituted or further substituted with R⁶, or
C₁-C₆ alkyl may also be substituted with C₃-C₈ cycloalkyl or with phenoxy which may be unsubstituted or substituted with R⁶ or with phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benxothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1,4-benzodioxanyl,
   each of which may be unsubstituted or substituted with R⁶, or

R⁵ is H, halo or C₁-C₆ alkyl optionally substituted with oxo; and

R⁶ is halo, CF₃, C₁-C₆ alkyl optionally substituted with oxo or hydroxy, or C₁-C₆ alkoxy optionally substituted with fluoro; or a pharmaceutically acceptable salt, ester prodrug, stereoisomer, diastereomer, enantiomer, racemate or a combination thereof.

R³ may be attached to the heterocyclic moiety of the compound of Formula I at either the 4 or 5 position (i.e., at either available carbon atom) and, accordingly, the remaining portion of the molecule will be attached at the remaining available carbon atom.

In some embodiments, the compound of Formula I has structure as described above and R is potassium, sodium, calcium, magnesium, lysine, choline or meglumine salt thereof.

In other embodiments, for the compound of Formula I, R is H, R¹ is H, R² is H, R⁵ is H, R³ is C₁-C₆ alkyl, X is O, and R⁴ is a phenyl, singularly or multiply substituted with R⁶, wherein R⁶ is halo, CF₃, C₁-C₆ alkoxyl or C₁-C₆ alkyl, or a pharmaceutically acceptable salt thereof.

In other embodiments, for the compound of Formula I, R is H, R¹ is H, R² is H, R⁵ is H, R³ is C₁-C₆ alkyl, X is O, and R⁴ is a phenyl, singularly or multiply substituted with R⁶, wherein R⁶ is halo, CF₃, C₁-C₆ alkoxyl or C₁-C₆ alkyl, and the stereochemistry at C-1′ is defined as S, or a pharmaceutically acceptable salt thereof.

In other embodiments, for the compound of Formula I, R is H, R¹ is H, R² is H, R⁵ is H, R³ is C₁-C₆ alkyl, X is S, and R⁴ is a phenyl, singularly or multiply substituted with R⁶, wherein R⁶ is halo, CF₃, C₁-C₆ alkoxyl or C₁-C₆ alkyl, and the stereochemistry at C-1′ is defined as S, or a pharmaceutically acceptable salt thereof.

In other embodiments, for the compound of Formula I, R is H, R¹ is H, R² is F, R⁵ is H, R³ is C₁-C₆ alkyl, X is O, and R⁴ is a phenyl, singularly or multiply substituted with R⁶, wherein R⁶ is halo, CF₃, C₁-C₆ alkoxyl or C₁-C₆ alkyl, and the stereochemistry at C-1′ is defined as S, or a pharmaceutically acceptable salt thereof.

In other embodiments, for the compound of Formula I, R is H, R¹ is H, R² is H, R⁵ is F, or R² and R⁵ are F, R³ is C₁-C₆ alkyl, X is O, and R⁴ is a phenyl, singularly or multiply substituted with R⁶, wherein R⁶ is halo, CF₃, C₁-C₆ alkoxyl or C₁-C₆ alkyl, and the stereochemistry at C-1′ is defined as S, or a pharmaceutically acceptable salt thereof.

In other embodiments, for the compound of Formula I, R is H, R¹ is H, R² is H, R⁵ is H, R³ is C₁-C₆ alkyl, X is O, and R⁴ is a phenyl, singularly or multiply substituted with R⁶, wherein R⁶ is halo, CF₃, C₁-C₆ alkoxyl or C₁-C₆ alkyl, and the stereochemistry at C-1′ is defined as R, or a pharmaceutically acceptable salt thereof.

In one embodiment, the compound of Formula I is either the free acid or the potassium, sodium, calcium, magnesium, lysine, choline or meglumine salt of one of the following structures:

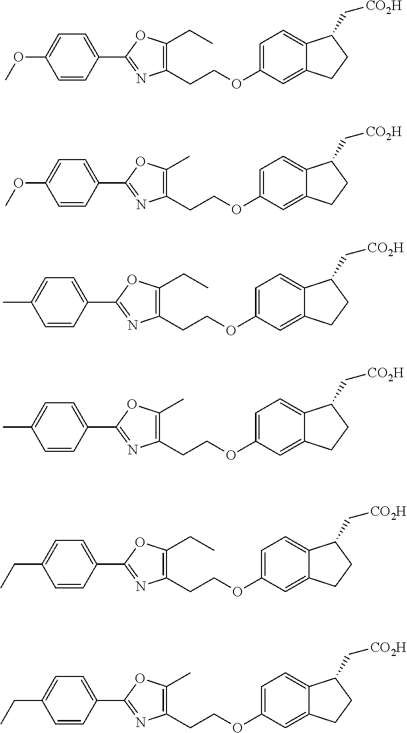 [SMILES: CCc1oc(-c2ccc(OC)cc2)nc1CCOc1ccc2c(c1)CC[C@H]2CC(=O)O ; COc1ccc(-c2nc(CCOc3ccc4c(c3)CC[C@H]4CC(=O)O)c(C)o2)cc1 ; CCc1oc(-c2ccc(C)cc2)nc1CCOc1ccc2c(c1)CC[C@H]2CC(=O)O ; Cc1ccc(-c2nc(CCOc3ccc4c(c3)CC[C@H]4CC(=O)O)c(C)o2)cc1 ; CCc1ccc(-c2nc(CCOc3ccc4c(c3)CC[C@H]4CC(=O)O)c(CC)o2)cc1 ; CCc1ccc(-c2nc(CCOc3ccc4c(c3)CC[C@H]4CC(=O)O)c(C)o2)cc1]

In another embodiment, the compound of Formula I is a the potassium or sodium salt of the structures:

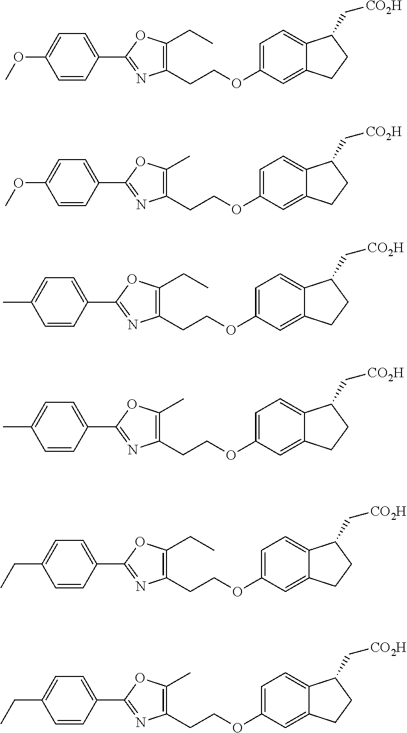 [SMILES: CCc1oc(-c2ccc(OC)cc2)nc1CCOc1ccc2c(c1)CC[C@H]2CC(=O)O ; COc1ccc(-c2nc(CCOc3ccc4c(c3)CC[C@H]4CC(=O)O)c(C)o2)cc1 ; CCc1oc(-c2ccc(C)cc2)nc1CCOc1ccc2c(c1)CC[C@H]2CC(=O)O ; Cc1ccc(-c2nc(CCOc3ccc4c(c3)CC[C@H]4CC(=O)O)c(C)o2)cc1 ; CCc1ccc(-c2nc(CCOc3ccc4c(c3)CC[C@H]4CC(=O)O)c(CC)o2)cc1 ; CCc1ccc(-c2nc(CCOc3ccc4c(c3)CC[C@H]4CC(=O)O)c(C)o2)cc1]

Exemplary compounds of Formula I are listed in Table 1 as the free acid, but may also be a pharmaceutically acceptable salt thereof.

TABLE 1
Illustrative Examples of Compounds of Formula I
Formula I

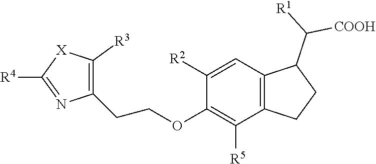

| Entry | | | | | | |
|---|---|---|---|---|---|---|
| No. | R¹ | R² | R³ | R⁴ | R⁵ | X |
| 1 | H | H | Me | Ph | H | O |
| 2 | H | H | Me | 2-F—Ph | H | O |
| 3 | H | H | Me | 2-Cl Ph | H | O |
| 4 | H | H | Me | 2-Me Ph | H | O |
| 5 | H | H | Me | 3-F—Ph | H | O |
| 6 | H | H | Me | 3-Cl Ph | H | O |
| 7 | H | H | Me | 3-CF₃ Ph | H | O |
| 8 | H | H | Me | 3-Me Ph | H | O |
| 9 | H | H | Me | 3-MeO Ph | H | O |
| 10 | H | H | Me | 4-F—Ph | H | O |
| 11 | H | H | Me | 4-Cl Ph | H | O |
| 12 | H | H | Me | 4-CF₃ Ph | H | O |
| 13 | H | H | Me | 4-Me Ph | H | O |
| 14 | H | H | Me | 4-Et Ph | H | O |
| 15 | H | H | Me | 4-MeO Ph | H | O |
| 16 | H | H | Me | 4-EtO Ph | H | O |
| 17 | H | H | Me | 2,3-di-F Ph | H | O |
| 18 | H | H | Me | 2,4-di-F Ph | H | O |
| 19 | H | H | Me | 3,4-di-F Ph | H | O |
| 20 | H | H | Me | 2,6-di-F Ph | H | O |
| 21 | H | H | Me | 2,3-di-Cl Ph | H | O |
| 22 | H | H | Me | 3,4-di-Cl Ph | H | O |
| 23 | H | H | Me | 2,4-di-Cl Ph | H | O |
| 24 | H | H | Me | 2,6-di-Cl Ph | H | O |
| 25 | H | H | Me | 2,3-di-Me Ph | H | O |
| 26 | H | H | Me | 2,4-di-Me Ph | H | O |
| 27 | H | H | Me | 3,4-di-Me Ph | H | O |
| 28 | H | H | Me | 2,6-di-Me Ph | H | O |
| 29 | H | H | Me | 2,3-di-MeO Ph | H | O |
| 30 | H | H | Me | 2,4-di-MeO Ph | H | O |
| 31 | H | H | Me | 3,4-di-MeO Ph | H | O |
| 32 | H | H | Et | Ph | H | O |
| 33 | H | H | Et | 2-Cl Ph | H | O |
| 34 | H | H | Et | 2-Me Ph | H | O |
| 35 | H | H | Et | 3-F—Ph | H | O |
| 36 | H | H | Et | 3-Cl Ph | H | O |
| 37 | H | H | Et | 3-CF₃ Ph | H | O |
| 38 | H | H | Et | 3-Me Ph | H | O |
| 39 | H | H | Et | 3-MeO Ph | H | O |
| 40 | H | H | Et | 4-F—Ph | H | O |
| 41 | H | H | Et | 4-Cl Ph | H | O |
| 42 | H | H | Et | 4-CF₃ Ph | H | O |
| 43 | H | H | Et | 4-Me Ph | H | O |
| 44 | H | H | Et | 4-Et Ph | H | O |
| 45 | H | H | Et | 4-MeO Ph | H | O |
| 46 | H | H | Et | 4-EtO Ph | H | O |
| 47 | H | H | Et | 2,3-di-F Ph | H | O |
| 48 | H | H | Et | 2,4-di-F Ph | H | O |
| 49 | H | H | Et | 3,4-di-F Ph | H | O |
| 50 | H | H | Et | 2,6-di-F Ph | H | O |
| 51 | H | H | Et | 2,3-di-Cl Ph | H | O |
| 52 | H | H | Et | 2,4-di-Cl Ph | H | O |
| 53 | H | H | Et | 3,4-di-Cl Ph | H | O |
| 54 | H | H | Et | 2,6-di-Cl Ph | H | O |
| 55 | H | H | Et | 2,3-di-Me Ph | H | O |
| 56 | H | H | Et | 2,4-di-Me Ph | H | O |
| 57 | H | H | Et | 3,4-di-Me Ph | H | O |
| 58 | H | H | Et | 2,6-di-Me Ph | H | O |
| 59 | H | H | Et | 2,3-di-MeO Ph | H | O |
| 60 | H | H | Et | 2,4-di-MeO Ph | H | O |
| 61 | H | H | Et | 3,4-di-MeO Ph | H | O |
| 62 | H | H | iPr | Ph | H | O |
| 63 | H | H | iPr | 2-F Ph | H | O |
| 64 | H | H | iPr | 2-Cl Ph | H | O |
| 65 | H | H | iPr | 2-Me Ph | H | O |
| 66 | H | H | iPr | 2-MeO Ph | H | O |
| 67 | H | H | iPr | 3-F—Ph | H | O |
| 68 | H | H | iPr | 3-Cl Ph | H | O |
| 69 | H | H | iPr | 3-CF₃ Ph | H | O |
| 70 | H | H | iPr | 3-Me Ph | H | O |
| 71 | H | H | iPr | 3-MeO Ph | H | O |
| 72 | H | H | iPr | 4-F—Ph | H | O |
| 73 | H | H | iPr | 4-Cl Ph | H | O |
| 74 | H | H | iPr | 4-CF₃ Ph | H | O |
| 75 | H | H | iPr | 4-Me Ph | H | O |
| 76 | H | H | iPr | 4-Et Ph | H | O |
| 77 | H | H | iPr | 4-MeO Ph | H | O |
| 78 | H | H | iPr | 4-EtO Ph | H | O |
| 79 | H | H | iPr | 2,3-di-F Ph | H | O |
| 80 | H | H | iPr | 2,4-di-F Ph | H | O |
| 81 | H | H | iPr | 3,4-di-F Ph | H | O |
| 82 | H | H | iPr | 2,3-di-F Ph | H | O |
| 83 | H | H | iPr | 2,3-di-Cl Ph | H | O |
| 84 | H | H | iPr | 2,4-di-Cl Ph | H | O |
| 85 | H | H | iPr | 2,6-di-Cl Ph | H | O |
| 86 | H | H | iPr | 3,4-di-Cl Ph | H | O |
| 87 | H | H | iPr | 2,3-di-Me Ph | H | O |
| 88 | H | H | iPr | 2,4-di-Me Ph | H | O |
| 89 | H | H | iPr | 2,3-di-Me Ph | H | O |
| 90 | H | H | iPr | 2,3-di-Me Ph | H | O |
| 91 | H | H | iPr | 2,3-di-MeO Ph | H | O |
| 92 | H | H | iPr | 2,4-di-MeO Ph | H | O |
| 93 | H | H | iPr | 3,4-di-MeO Ph | H | O |
| 94 | Me | H | Et | 4-MeO Ph | H | O |
| 95 | Me | H | Et | 4-MeO Ph | H | S |
| 96 | H | H | Et | 4-MeO Ph | H | S |
| 97 | H | H | Me | 4-Et Ph | H | S |
| 98 | H | F | Et | 4-MeO Ph | H | O |
| 99 | H | H | Et | 4-MeO Ph | F | O |
| 100 | H | H | Et | 4-F-Ph | H | S |
| 101 | H | H | Et | 4-Cl Ph | H | S |
| 102 | H | H | Et | 4-CF₃ Ph | H | S |
| 103 | H | H | Et | 4-Me Ph | H | S |
| 104 | H | H | Et | 4-MeO Ph | H | S |
| 105 | H | H | Et | 4-EtO Ph | H | S |

The particular process to be utilized in the preparation of the compounds of this invention depends upon the specific compound desired. Such factors as the selection of the specific X moiety, and the specific substituents possible at various locations on the molecule, all play a role in the path to be followed in the preparation of the specific compounds of this invention. Those factors are readily recognized by one of ordinary skill in the art.

In general, the compounds of this invention may be prepared by standard techniques known in the art and by known processes analogous thereto. For example, the compounds may be prepared according to methods described in U.S. Pat. No. 6,828,335, and U.S. application Ser. No. 13/375,878, which are incorporated by reference in its entirety. The present invention also encompasses indane acetic acid compounds and derivatives described in U.S. Pat. No. 7,112,597 in U.S. Pat. No. 8,541,618, and in U.S. Pat. No. 8,552,203, which are incorporated by references in their entirety. The present invention also encompasses indane acetic acid derivatives and their use described in US application publication number 2014/0086910, and in U.S. patent application Ser. No. 14/477,114, which are incorporated by references in their entirety.

A salt of a compound described in the present invention may be prepared in situ during the final isolation and purification of a compound or by separately reacting the purified compound in its free base form with a suitable organic or inorganic acid and isolating the salt thus formed. Likewise, when the compound described in the present invention contains a carboxylic acid moiety, (e.g., R═H), a salt of said compound may be prepared by separately reacting it with a suitable inorganic or organic base and isolating the salt thus formed. The term “pharmaceutically acceptable salt” refers to a relatively non-toxic, inorganic or organic acid addition salt of a compound of the present invention (see, e.g., Berge et al., J. Pharm. Sci. 66:1-19, 1977).

Representative salts of the compounds described in the present invention include the conventional non-toxic salts and the quaternary ammonium salts, which are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, tartrate, thiocyanate, tosylate, undecanoate, and the like.

Base salts include, for example, alkali metal salts such as potassium and sodium salts, alkaline earth metal salts such as calcium and magnesium salts, and ammonium salts with organic bases such as dicyclohexylamine and N-methyl-D-glucamine. Additionally, basic nitrogen containing groups in the conjugate base may be quaternized with alkyl halides, e.g., C₁₋₉ alkyl halides such as methyl, ethyl, propyl, and butyl chlorides, bromides and iodides; dialkyl sulfates like dimethyl, diethyl, and dibutyl sulfate; and diamyl sulfates, C₁₀₋₄₀ alkyl halides such as decyl, lauryl, myristyl and strearyl chlorides, bromides and iodides; or aralkyl halides like benzyl and phenethyl bromides. In some embodiments, the salts are alkali salt such as sodium or potassium salt or an adduct with an acceptable nitrogen base such as meglumine (N-Methyl-d-glucamine) salt.

The esters of the compounds described in the present invention are non-toxic, pharmaceutically acceptable esters, for example, alkyl esters such as methyl, ethyl, propyl, isopropyl, butyl, isobutyl, or pentyl esters. Additional esters such as, for example, methyl ester or phenyl-C₁-C₅ alkyl may be used. The compound described in the present invention may be esterified by a variety of conventional procedures including reacting the appropriate anhydride, carboxylic acid, or acid chloride with the alcohol group of the compounds described in the present invention compound. The appropriate anhydride may be reacted with the alcohol in the presence of a base to facilitate acylation such as 1,8-bis[dimethylamino]naphthalene or N,N-dimethylaminopyridine. An appropriate carboxylic acid may be reacted with the alcohol in the presence of a dehydrating agent such as dicyclohexylcarbodiimide, 1-[3-dimethylaminopropyl]-3-ethylcarbodiimide, or other water soluble dehydrating agents which are used to drive the reaction by the removal of water, and optionally, an acylation catalyst. Esterification may also be effected using the appropriate carboxylic acid in the presence of trifluoroacetic anhydride and optionally, pyridine, or in the presence of N, N-carbonyldiimidazole with pyridine. Reaction of an acid chloride with the alcohol may be carried out with an acylation catalyst such as 4-DMAP or pyridine.

One skilled in the art would readily know how to successfully carry out these, as well as other methods of esterification of alcohols.

Additionally, sensitive or reactive groups on the compound described in the present invention may need to be protected and deprotected during any of the above methods for forming esters. Protecting groups in general may be added and removed by conventional methods well known in the art (see, e.g., T. W. Greene and P.G.M. Wuts, Protective Groups in Organic Synthesis; Wiley: New York, (1999)).

The compounds described in the present invention may contain one or more asymmetric centers, depending upon the location and nature of the various substituents desired. Asymmetric carbon atoms may be present in the (R) or (S) configuration. Preferred isomers are those with the absolute configuration, which produces the compound of described in the present invention with the more desirable biological activity. In certain instances, asymmetry may also be present due to restricted rotation about a given bond, for example, the central bond adjoining two aromatic rings of the specified compounds.

Substituents on a ring may also be present in either cis or trans form, and a substituent on a double bond may be present in either Z or E form.

It is intended that all isomers (including enantiomers and diastereomers), either by nature of asymmetric centers or by restricted rotation as described above, as separated, pure or partially purified isomers or racemic mixtures thereof, be included within the scope of the instant invention. The purification of said isomers and the separation of said isomeric mixtures may be accomplished by standard techniques known in the art.

As described herein, compounds of the invention may optionally be substituted with one or more substituents, such as are illustrated generally above, or as exemplified by particular classes, subclasses, and species of the invention. In general, the term “substituted” refers to the replacement of hydrogen radicals in a given structure with the radical of a specified substituent. Unless otherwise indicated, a substituted group may have a substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

### C. EVALUATION OF BIOLOGICAL ACTIVITY OF COMPOUNDS

PPAR receptor agonist activity may be determined by conventional screening methods known to the skilled in the art. For example, methods described in U.S. Patent Application Publication No. 2007/0054907, 2008/0262047 and U.S. Pat. No. 7,314,879, which are incorporated by reference in their entireties.

### D. NASH/NAFLD Animal Model

The compounds described in the present invention may be tested in any animal model known to those skilled in the art. Exemplary animal models of NASH/NAFLD include, but are not limited to, transgenic mouse models and dietary rodent models such as the Long Evans rat high fat diet model (See: Takahashi, Y., et al. Animal models of nonalcoholic fatty liver disease/nonalcoholic steatohepatitis. World J Gastroenterol 2012 May 21; 18(19): 2300-2308.) Compound testing in the present invention can be carried out using the following Long Evans rat high fat diet NASH/NAFLD model methodology. Adult Long Evans male rats (n=6 per group) are pair-fed for 8 weeks with high fat (HFD) or low fat (LFD) chow diets. Drug therapies can be administered by oral gavage q.d. for the last 5 weeks of the 8-week model. The HFD supplies 60% of the kcal in fat (54% from lard, 6% from soybean oil), 20% in carbohydrates, and 20% in protein, whereas the LFD supplies 10% of the kcal in fat (4.4% from lard, 5.6% from soybean oil), 70% in carbohydrates, and 20% in protein.

For each model, the test result is compared with a control group that is not treated with the compounds described in the present invention. The treated animals are expected to demonstrate significant improvement in the performance of a variety of tests that measure steatosis, inflammation, fibrosis, dyslipidemia, and insulin resistance.

### E. Pharmaceutical Compositions

According to another aspect of the present invention, pharmaceutical compositions of compounds described herein are provided. In some embodiments, the pharmaceutical compositions further include a pharmaceutically acceptable carrier.

In some embodiments, the pharmaceutical compositions described herein may further include one or more additional therapeutic agents.

In one embodiment, the additional therapeutic agents are used to treat or prevent NASH/NAFLD as well as the following diseases:

### 1. Farber's Disease

2. ACLF (acute-on-chronic liver failure)
3. CLF (chronic liver failure)
4. POLT-HCV-SVR post-orthotopic liver transplant, or POLT, due to hepatitis C virus, or HCV, infection and have subsequently achieved sustained viral response, or SVR, following anti-HCV therapy
5. Alagille syndrome
6. Progressive Familial Intrahepatic cholestasis (PFIC)

### 7. Primary Biliary Cirrhosis (PBC)

### 8. Primary Sclerosing Cholangitis

### 9. Autosomal Dominant Polycystic Liver Disease (ADPCLD)

10. Treatment of liver transplant patients with reestablished fibrosis

### 11. Cholesteryl Ester Storage Disease (CESD)

### 12. Severe Hypertriglyceridemia (SHTG)

### 13. Homozygous Familial Hypercholesterolemia (HoFH)

### 14. Hepatic Encephalopathy (HE)

### 15. Alcoholic Liver Disease

Exemplary additional therapeutic agents include, but are not limited to combination with: farnesoid X receptor agonists such as obeticholic acid and Px-104, aramchol, GR-MD-02, cysteamine bitartrate, simtuzumab, emricasan, GFT-505, CER-002, KD3010, KD3020, MBX8025, LUM002, RP-103, galectin-3 blockers such as LIPC-1010 and GR-MD-02, cenicriviroc, vascular adhesion protein-1 inhibitors such as PXS4728A, metformin, PPAR gamma agonists such as rosiglitazone and pioglitazone, metformin, pentoxyfylline, vitamin E, selenium, omega-3 fatty acids and betaine

Based on well-known assays used to determine the efficacy for treatment of conditions identified above in mammals, and by comparison of these results with the results of known medicaments that are used to treat these conditions, the effective dosage of the compounds of this invention can readily be determined for treatment of each desired indication. The amount of the active ingredient (e.g., compounds) to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered may generally range from about 0.0001 mg/kg to about 10 mg/kg, and preferably from about 0.001 mg/kg to about 10 mg/kg body weight per day. A unit dosage may contain from about 0.05 mg to about 500 mg of active ingredient, and may be administered one or more times per day. The daily dosage for administration by injection, including intravenous, intramuscular, subcutaneous, and parenteral injections, and use of infusion techniques may be from about 0.0001 mg/kg to about 10 mg/kg. The daily rectal dosage regimen may be from 0.0001 mg/kg to 10 mg/kg of total body weight. The transdermal concentration may be that required to maintain a daily dose of from 0.0001 mg/kg to 10 mg/kg.

Of course, the specific initial and continuing dosage regimen for each patient will vary according to the nature and severity of the condition as determined by the attending diagnostician, the activity of the specific compound employed, the age of the patient, the diet of the patient, time of administration, route of administration, rate of excretion of the drug, drug combinations, and the like. The desired mode of treatment and number of doses of a compound of the present invention may be ascertained by those skilled in the art using conventional treatment tests.

The compounds of this invention may be utilized to achieve the desired pharmacological effect by administration to a patient in need thereof in an appropriately formulated pharmaceutical composition. A patient, for the purpose of this invention, is a mammal, including a human, in need of treatment for a particular condition or disease. Therefore, the present invention includes pharmaceutical compositions which include a pharmaceutically acceptable carrier and a therapeutically effective amount of a compound. A pharmaceutically acceptable carrier is any carrier which is relatively non-toxic and innocuous to a patient at concentrations consistent with effective activity of the active ingredient so that any side effects ascribable to the carrier do not vitiate the beneficial effects of the active ingredient. A therapeutically effective amount of a compound is that amount which produces a result or exerts an influence on the particular condition being treated. The compounds described herein may be administered with a pharmaceutically-acceptable carrier using any effective conventional dosage unit forms, including, for example, immediate and timed release preparations, orally, parenterally, topically, or the like.

For oral administration, the compounds may be formulated into solid or liquid preparations such as, for example, capsules, pills, tablets, troches, lozenges, melts, powders, solutions, suspensions, or emulsions, and may be prepared according to methods known to the art for the manufacture of pharmaceutical compositions. The solid unit dosage forms may be a capsule which can be of the ordinary hard- or soft-shelled gelatin type containing, for example, surfactants, lubricants, and inert fillers such as lactose, sucrose, calcium phosphate, and corn starch.

In another embodiment, the compounds of this invention may be tableted with conventional tablet bases such as lactose, sucrose, and cornstarch in combination with binders such as acacia, cornstarch, or gelatin; disintegrating agents intended to assist the break-up and dissolution of the tablet following administration such as potato starch, alginic acid, corn starch, and guar gum; lubricants intended to improve the flow of tablet granulation and to prevent the adhesion of tablet material to the surfaces of the tablet dies and punches, for example, talc, stearic acid, or magnesium, calcium or zinc stearate; dyes; coloring agents; and flavoring agents intended to enhance the aesthetic qualities of the tablets and make them more acceptable to the patient. Suitable excipients for use in oral liquid dosage forms include diluents such as water and alcohols, for example, ethanol, benzyl alcohol, and polyethylene alcohols, either with or without the addition of a pharmaceutically acceptable surfactant, suspending agent, or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent, and one or more preservatives. Suitable dispersing or wetting agents and suspending agents are exemplified by those already mentioned above. Additional excipients, for example, those sweetening, flavoring and coloring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soybean and lecithin, (3) esters or partial esters derived from fatty acids and hexitol anhydrides, for example, sorbitan monooleate, and (4) condensation products of said partial esters with ethylene oxide, for example, polyoxyethylene sorbitan monooleate. The emulsions may also contain sweetening and flavoring agents.

Oily suspensions may be formulated by suspending the active ingredient in a vegetable oil such as, for example, arachis oil, olive oil, sesame oil, or coconut oil; or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs may be formulated with sweetening agents such as, for example, glycerol, propylene glycol, sorbitol, or sucrose. Such formulations may also contain a demulcent, and preservative, flavoring and coloring agents.

The compounds of this invention may also be administered parenterally, that is, subcutaneously, intravenously, intramuscularly, or interperitoneally, as injectable dosages of the compound in a physiologically acceptable diluent with a pharmaceutical carrier which may be a sterile liquid or mixture of liquids such as water, saline, aqueous dextrose and related sugar solutions; an alcohol such as ethanol, isopropanol, or hexadecyl alcohol; glycols such as propylene glycol or polyethylene glycol; glycerol ketals such as 2,2-dimethyl-1,1-dioxolane-4-methanol, ethers such as poly(ethyleneglycol) 400; an oil; a fatty acid; a fatty acid ester or glyceride; or an acetylated fatty acid glyceride with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum, and mineral oil. Suitable fatty acids include oleic acid, stearic acid, and isostearic acid. Suitable fatty acid esters are, for example, ethyl oleate and isopropyl myristate. Suitable soaps include fatty alkali metal, ammonium, and triethanolamine salts and suitable detergents include cationic detergents, for example, dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; nonionic detergents, for example, fatty amine oxides, fatty acid alkanolamides, and polyoxyethylenepolypropylene copolymers; and amphoteric detergents, for example, alkyl-beta-aminopropionates, and 2-alkylimidazoline quarternary ammonium salts, as well as mixtures.

The parenteral compositions of this invention may typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of surfactant in such formulation ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadecaethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, and isotonic sodium chloride solution. In addition, sterile fixed oils are conventionally employed as solvents or suspending media. For this purpose, any bland, fixed oil may be employed including synthetic mono or diglycerides. In addition, fatty acids such as oleic acid may be used in the preparation of injectables.

A composition of the invention may also be administered in the form of suppositories for rectal administration of the drug. These compositions may be prepared by mixing the drug (e.g., compound) with a suitable non-irritation excipient which is solid at ordinary temperatures but liquid at the rectal temperature and will therefore melt in the rectum to release the drug. Such materials are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, incorporated herein by reference). Such patches may be constructed for continuous, pulsatile, or on demand delivery of pharmaceutical agents.

It may be desirable or necessary to introduce the pharmaceutical composition to the patient via a mechanical delivery device. The construction and use of mechanical delivery devices for the delivery of pharmaceutical agents is well known in the art. For example, direct techniques for administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, incorporated herein by reference.

The compositions of the invention may also contain other conventional pharmaceutically acceptable compounding ingredients, generally referred to as carriers or diluents, as necessary or desired. Any of the compositions of this invention may be preserved by the addition of an antioxidant such as ascorbic acid or by other suitable preservatives. Conventional procedures for preparing such compositions in appropriate dosage forms can be utilized.

Commonly used pharmaceutical ingredients which may be used as appropriate to formulate the composition for its intended route of administration include: acidifying agents, for example, but are not limited to, acetic acid, citric acid, fumaric acid, hydrochloric acid, nitric acid; and alkalinizing agents such as, but are not limited to, ammonia solution, ammonium carbonate, diethanolamine, monoethanolamine, potassium hydroxide, sodium borate, sodium carbonate, sodium hydroxide, triethanolamine, or trolamine.

Other pharmaceutical ingredients include, for example, but are not limited to, adsorbents (e.g., powdered cellulose and activated charcoal); aerosol propellants (e.g., carbon dioxide, CCl₂F₂, F₂ClC—CClF₂ and CClF₃); air displacement agents (e.g., nitrogen and argon); antifungal preservatives (e.g., benzoic acid, butylparaben, ethylparaben, methylparaben, propylparaben, sodium benzoate); antimicrobial preservatives (e.g., benzalkonium chloride, benzethonium chloride, benzyl alcohol, cetylpyridinium chloride, chlorobutanol, phenol, phenylethyl alcohol, phenylmercuric nitrate and thimerosal); antioxidants (e.g., ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite); binding materials (e.g., block polymers, natural and synthetic rubber, polyacrylates, polyurethanes, silicones and styrene-butadiene copolymers); buffering agents (e.g., potassium metaphosphate, potassium phosphate monobasic, sodium acetate, sodium citrate anhydrous and sodium citrate dihydrate); carrying agents (e.g., acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection); chelating agents (e.g., edetate disodium and edetic acid); colorants (e.g., FD&C Red No. 3, FD&C Red No. 20, FD&C Yellow No. 6, FD&C Blue No. 2, D&C Green No. 5, D&C Orange No. 5, D&C Red No. 8, caramel and ferric oxide red); clarifying agents (e.g., bentonite); emulsifying agents (includes but are not limited to, acacia, cetomacrogol, cetyl alcohol, glyceryl monostearate, lecithin, sorbitan monooleate, polyethylene 50 stearate); encapsulating agents (e.g., gelatin and cellulose acetate phthalate); flavorants (e.g., anise oil, cinnamon oil, cocoa, menthol, orange oil, peppermint oil and vanillin); humectants (e.g., glycerin, propylene glycol and sorbitol); levigating agents (e.g., mineral oil and glycerin); oils (e.g., arachis oil, mineral oil, olive oil, peanut oil, sesame oil and vegetable oil); ointment bases (e.g., lanolin, hydrophilic ointment, polyethylene glycol ointment, petrolatum, hydrophilic petrolatum, white ointment, yellow ointment, and rose water ointment); penetration enhancers (transdermal delivery) (e.g., monohydroxy or polyhydroxy alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas); plasticizers (e.g., diethyl phthalate and glycerin); solvents (e.g., alcohol, corn oil, cottonseed oil, glycerin, isopropyl alcohol, mineral oil, oleic acid, peanut oil, purified water, water for injection, sterile water for injection and sterile water for irrigation); stiffening agents (e.g., cetyl alcohol, cetyl esters wax, microcrystalline wax, paraffin, stearyl alcohol, white wax and yellow wax); suppository bases (e.g., cocoa butter and polyethylene glycols (mixtures)); surfactants (e.g., benzalkonium chloride, nonoxynol 10, oxtoxynol 9, polysorbate 80, sodium lauryl sulfate and sorbitan monopalmitate); suspending agents (e.g., agar, bentonite, carbomers, carboxymethylcellulose sodium, hydroxyethyl cellulose, hydroxypropyl cellulose, hydroxypropyl methylcellulose, kaolin, methylcellulose, tragacanth and veegum); sweetening e.g., aspartame, dextrose, glycerin, mannitol, propylene glycol, saccharin sodium, sorbitol and sucrose); tablet anti-adherents (e.g., magnesium stearate and talc); tablet binders (e.g., acacia, alginic acid, carboxymethylcellulose sodium, compressible sugar, ethylcellulose, gelatin, liquid glucose, methylcellulose, povidone and pregelatinized starch); tablet and capsule diluents (e.g., dibasic calcium phosphate, kaolin, lactose, mannitol, microcrystalline cellulose, powdered cellulose, precipitated calcium carbonate, sodium carbonate, sodium phosphate, sorbitol and starch); tablet coating agents (e.g., liquid glucose, hydroxyethyl cellulose, hydroxypropyl cellulose, hydroxypropyl methylcellulose, methylcellulose, ethylcellulose, cellulose acetate phthalate and shellac); tablet direct compression excipients (e.g., dibasic calcium phosphate); tablet disintegrants (e.g., alginic acid, carboxymethylcellulose calcium, microcrystalline cellulose, polacrillin potassium, sodium alginate, sodium starch glycollate and starch); tablet glidants (e.g., colloidal silica, corn starch and talc); tablet lubricants (e.g., calcium stearate, magnesium stearate, mineral oil, stearic acid and zinc stearate); tablet/capsule opaquants (e.g., titanium dioxide); tablet polishing agents (e.g., carnuba wax and white wax); thickening agents (e.g., beeswax, cetyl alcohol and paraffin); tonicity agents (e.g., dextrose and sodium chloride); viscosity increasing agents (e.g., alginic acid, bentonite, carbomers, carboxymethylcellulose sodium, methylcellulose, povidone, sodium alginate and tragacanth); and wetting agents (e.g., heptadecaethylene oxycetanol, lecithins, polyethylene sorbitol monooleate, polyoxyethylene sorbitol monooleate, and polyoxyethylene stearate).

The compounds described herein may be administered as the sole pharmaceutical agent or in combination with one or more other pharmaceutical agents where the combination causes no unacceptable adverse effects. For example, compounds of this invention can be combined with known anti-oxidants, anti-obesity agents, insulin sensitizers, anti-fibrotics, anti-dyslipidemics, and the like, as well as with admixtures and combinations thereof.

The compounds described herein may also be utilized, in free base form or in compositions, in research and diagnostics, or as analytical reference standards, and the like. Therefore, the present invention includes compositions which include an inert carrier and an effective amount of a compound identified by the methods described herein, or a salt or ester thereof. An inert carrier is any material which does not interact with the compound to be carried and which lends support, means of conveyance, bulk, traceable material, and the like to the compound to be carried. An effective amount of compound is that amount which produces a result or exerts an influence on the particular procedure being performed.

The compounds may be administered to subjects by any suitable route, including orally (inclusive of administration via the oral cavity), parenterally, by inhalation spray, topically, transdermally, rectally, nasally, sublingually, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, parenterally, transdermally or by inhalation spray.

It should also be understood that a specific dosage and treatment regimen for any particular patient will depend upon a variety of factors, including the activity of the specific compound employed, the age, body weight, general health, gender, diet, time of administration, rate of excretion, drug combination, and the judgment of the treating physician and the severity of the particular disease being treated. The amount of a compound of the present invention in the composition will also depend upon the particular compound in the composition.

The following examples are presented to illustrate the invention described herein, but should not be construed as limiting the scope of the invention in any way.

Capsule Formulation

A capsule formula is prepared from:

| | |
|---|---|
| Compound of this invention | 10 mg |
| Starch | 109 mg |
| Magnesium stearate | 1 mg |

The components are blended, passed through an appropriate mesh sieve, and filled into hard gelatin capsules.

Tablet Formulation

A tablet is prepared from:

| | |
|---|---|
| Compound of this invention | 25 mg |
| Cellulose, microcrystalline | 200 mg |
| Colloidal silicon dioxide | 10 mg |
| Stearic acid | 5.0 mg |

The ingredients are mixed and compressed to form tablets. Appropriate aqueous and non-aqueous coatings may be applied to increase palatability, improve elegance and stability or delay absorption.

Sterile IV Solution

A mg/mL solution of the desired compound of this invention is made using sterile, injectable water, and the pH is adjusted if necessary. The solution is diluted for administration with sterile 5% dextrose and is administered as an IV infusion.

Intramuscular Suspension

The following intramuscular suspension is prepared:

| | |
|---|---|
| Compound of this invention | 50 mg/mL |
| Sodium carboxymethylcellulose | 5 mg/mL |
| TWEEN 80 | 4 mg/mL |
| Sodium chloride | 9 mg/mL |
| Benzyl alcohol | 9 mg/mL |

The suspension is administered intramuscularly.

Hard Shell Capsules

A large number of unit capsules are prepared by filling standard two-piece hard galantine capsules each with powdered active ingredient, 150 mg of lactose, 50 mg of cellulose, and 6 mg of magnesium stearate.

Soft Gelatin Capsules

A mixture of active ingredients in a digestible oil, such as soybean oil, cottonseed oil, or olive oil, is prepared and injected by means of a positive displacement pump into molten gelatin to form soft gelatin capsules containing the active ingredient. The capsules are washed and dried. The active ingredient can be dissolved in a mixture of polyethylene glycol, glycerin and sorbitol to prepare a water miscible medicine mix.

Immediate Release Tablets/Capsules

These are solid oral dosage forms made by conventional and novel processes. These units are taken orally without water for immediate dissolution and delivery of the medication. The active ingredient is mixed in a liquid containing ingredient such as sugar, gelatin, pectin, and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

### F. METHODS OF USE

NASH/NAFLD and other cited diseases including:

Farber's Disease

ACLF (acute-on-chronic liver failure)

CLF (chronic liver failure)

POLT-HCV-SVR post-orthotopic liver transplant, or POLT, due to hepatitis C virus, or HCV, infection and have subsequently achieved sustained viral response, or

SVR, following anti-HCV therapy

Alagille syndrome

Progressive Familial Intrahepatic cholestasis (PFIC)

Primary Biliary Cirrhosis (PBC)

Primary Sclerosing Cholangitis

Autosomal Dominant Polycystic Liver Disease (ADPCLD)

Treatment of liver transplant patients with reestablished fibrosis

Cholesteryl Ester Storage Disease (CESD)

Severe Hypertriglyceridemia (SHTG)

Homozygous Familial Hypercholesterolemia (HoFH)

Hepatic Encephalopathy (HE)

Alcoholic Liver Disease

According to one aspect of the present invention, methods of preventing or treating NASH/NAFLD and the remaining cited conditions are provided. The methods include administering to a subject in need of such treatment an effective amount of a compound of the present invention. In some embodiments, the compound is administered intravenously, orally, buccally, transdermally, rectally, nasally, optically, intrathecally, or intra-cranially.

In another embodiment, the compounds of the present invention may be administered in combination with one or more additional therapeutic agent. Exemplary additional therapeutic agents include, but are not limited to farnesoid X receptor agonists such as obeticholic acid and Px-104, aramchol, GR-MD-02, cysteamine bitartrate, simtuzumab, emricasan GFT-505, CER-002, KD3010, KD3020, MBX8025, LUM002, RP-103, galectin-3 blockers such as LIPC-1010 and GR-MD-02, cenicriviroc, vascular adhesion protein-1 inhibitors such as PXS4728A, metformin, PPAR gamma agonists such as rosiglitazone and pioglitazone, pentoxyfylline, vitamin E, selenium, omega-3 fatty acids and betaine. The compounds described herein may be administered in combination with one or more further medicaments of use for the treatment or prevention of the listed conditions and disease.

Depending on the individual medicaments utilized in a combination therapy for simultaneous administration, they may be formulated in combination (where a stable formulation may be prepared and where desired dosage regimes are compatible) or the medicaments may be formulated separately (for concomitant or separate administration through the same or alternative routes).

In some embodiments, the subject of the present invention possesses one or more risk factors for developing disease selected from a family history of the disease; Obesity, Insulin resistance and Type 2 Diabetes, High cholesterol, High triglycerides and Metabolic syndrome.

### G. EXAMPLES

Embodiments of the present invention will now be described by way of example only with respect to the following non-limiting examples.

In general, the compounds of this invention may be prepared by standard techniques known in the art and by known processes analogous thereto. For example, the compounds may be prepared according to methods described in U.S. Pat. No. 6,828,335, and U.S. application Ser. No. 13/375,878 which are incorporated by reference in its entirety.

### Example 1

### Ethyl [(1S)-5-hydroxy-2,3-dihydro-1H-inden-1-yl]acetate

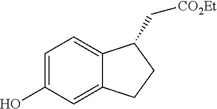 [SMILES: CCOC(=O)C[C@@H]1CCc2cc(O)ccc21]

Prepared in six steps from 5-methoxy indanone as described in U.S. Pat. No. 6,828,3335.

### Example 2

### 2-[5-ethyl-2-(4-methoxyphenyl)-1,3-oxazol-4-yl]ethanol

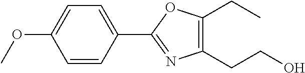 [SMILES: CCc1oc(-c2ccc(OC)cc2)nc1CCO]

Prepared from L-aspartic acid β-methyl ester hydrochloride, 4-methoxy benzoyl chloride and proprionic anhydride as generally described in U.S. Pat. No. 6,828,3335.

### Example 3

### 2-[2-(4-methoxyphenyl)-5-methyl-1,3-oxazol-4-yl]ethanol

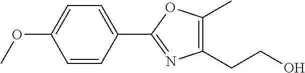 [SMILES: COc1ccc(-c2nc(CCO)c(C)o2)cc1]

Prepared from L-aspartic acid β-methyl ester hydrochloride, 4-methoxy benzoyl chloride and acetic anhydride as generally described in U.S. Pat. No. 6,828,335.

### Example 4

### 2-[5-Ethyl-2-(4-methylphenyl)-1,3-oxazol-4-yl]ethanol

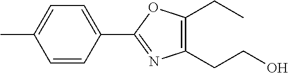 [SMILES: CCc1oc(-c2ccc(C)cc2)nc1CCO]

Prepared from L-aspartic acid β-methyl ester hydrochloride, p-toluoyl chloride and proprionic anhydride as generally described in U.S. Pat. No. 6,828,335.

### Example 5

### 2-[5-Methyl-2-(4-methylphenyl)-1,3-oxazol-4-yl]ethanol

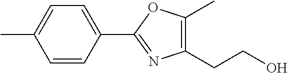 [SMILES: Cc1ccc(-c2nc(CCO)c(C)o2)cc1]

Prepared as from L-aspartic acid β-methyl ester hydrochloride, p-toluoyl chloride and acetic anhydride as described in U.S. Pat. No. 6,828,335.

### Example 6

### 2-[5-Ethyl-2-(4-ethylphenyl)-1,3-oxazol-4-yl]ethanol

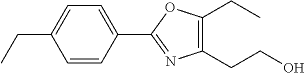 [SMILES: CCc1ccc(-c2nc(CCO)c(CC)o2)cc1]

Prepared from L-aspartic acid β-methyl ester hydrochloride, 4-ethyl benzoyl chloride and proprionic anhydride as generally described in U.S. Pat. No. 6,828,335.

### Example 7

### 2-[2-(4-Ethylphenyl)-5-methyl-1,3-oxazol-4-yl]ethanol

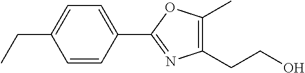 [SMILES: CCc1ccc(-c2nc(CCO)c(C)o2)cc1]

Prepared from L-aspartic acid β-methyl ester hydrochloride, 4-ethyl benzoyl chloride and acetic anhydride as generally described in U.S. Pat. No. 6,828,335.

### Example 8

### 2-(5-ethyl-2-(4-methoxyphenyl)oxazol-4-yl)ethyl benzenesulfonate

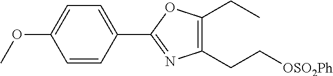

The intermediate from Example 2 (400.8 g), 15.0 g trimethylamine hydrochloride and 3.2 L dichloromethane was added to a 22 L reactor. The reaction mixture was stirred and cooled to 3.8° C. 680 mL of triethylamine was then added to the reactor. Benzenesulfonyl chloride (400 g) is slowly added to the reactor while maintaining the temperature below 12° C. The reaction was cooled to between 5° C. and 10° C. for three hours and then heated to 20° C. The contents of the reactor were stirred overnight at 24° C. Additional 3.2 L of dichloromethane was added to the reactor. The mixture was cooled to 5.0° C. and 205 mL 3-dimethylamino-1-propylamine was added. The mixture is stirred at 4.8° C. for 16 minutes. An aqueous citric acid solution (3 L of 1M) was slowly added to the reactor so as to maintain the temperature below 16° C. The resulting mixture was heated to 20° C. and stirred for 10 minutes. The phases were separated, and the organics were washed with 3 L of 1M citric acid solution, 3 L saturated sodium bicarbonate solution, 3 L brine solution, dried with magnesium sulfate, filtered and concentrated. The residue was treated with n-heptane and concentrated to give 542 g of crude 2-(5-ethyl-2-(4-methoxyphenyl)oxazol-4-yl)ethyl benzenesulfonate.

### Example 9

### (S)-Ethyl 2-(5-(2-(5-ethyl-2-(4-methoxyphenyl)oxazol-4-yl)ethoxy)-2,3-dihydro-1H-inden-1-yl)acetate

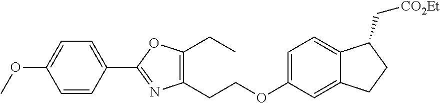 [SMILES: CCOC(=O)C[C@@H]1CCc2cc(OCCc3nc(-c4ccc(OC)cc4)oc3CC)ccc21]

A 22 L reactor was charged with 302.3 g of ethyl [(1S)-5-hydroxy-2,3-dihydro-1H-inden-1-yl]acetate (Example 1), 539.3 g crude 2-(5-ethyl-2-(4-methoxyphenyl)oxazol-4-yl)ethyl benzenesulfonate (Example 8) and 3.4 L acetonitrile. The mixture was stirred until all of the solids dissolved; then, 670.6 g cesium carbonate was added. The mixture is heated to 70° C. and held 16 hours. An additional charge of 60.2 g of compound from Example 1 was added to the reactor. The mixture was heated to 70° C. for one hour and additional cesium carbonate (316.9 g) was added and heating was continued for 2.5 hours at 70° C. The reaction mixture was cooled to 24° C. and 4 L n-heptane, 2.4 L USP water, 2.4 L brine solution and 4 L ethyl acetate was charged to the reactor. The biphasic mixture was stirred for 5 minutes, then allowed to separate. The organic layer was washed with 2×2.4 L 5% sodium hydroxide solution and 2.4 L USP water, and 2.4 L brine. The solvent is removed via rotary evaporation until solids precipitate. Addition of 7.7 L n-heptane and stirring produced a slurry, which was filtered, and the filter cake was rinsed with the filtrate and then with 2.4 L n-heptane. The product air dried and then dried in a vacuum oven at 40° C. to give (S)-ethyl 2-(5-(2-(5-ethyl-2-(4-methoxyphenyl)oxazol-4-yl)ethoxy)-2,3-dihydro-1H-inden-1-yl)acetate as an off white solid.

### Example 10

### (S)-2-(5-(2-(5-ethyl-2-(4-methoxyphenyl)oxazol-4-yl)ethoxy)-2,3-dihydro-1H-inden-1-yl)acetic acid

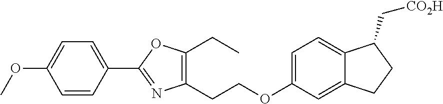 [SMILES: CCc1oc(-c2ccc(OC)cc2)nc1CCOc1ccc2c(c1)CC[C@H]2CC(=O)O]

A 22 L flask was charged with 478.9 g of (S)-ethyl 2-(5-(2-(5-ethyl-2-(4-methoxyphenyl)oxazol-4-yl)ethoxy)-2,3-dihydro-1H-inden-1-yl)acetate (Example 9) and 1.2 L ethanol and cooled to 20° C. To the 22 L flask was charged 1.6 L of 1N sodium hydroxide solution. The reaction mixture was heated to 65° C. for 30, then cooled to 25° C., and concentrated to an oil. A new reaction flask was charged with 4.8 L USP water and 1.9 L 1N hydrochloric acid solution, vigorously stirred and cooled to 23° C. The product oil was added to the solution via an addition funnel. The resulting suspension is stirred at approximately 23° C., and the pH is checked: 1.6 (target ≤2). The solids were filtered and then washed with the mother liquor. The solids were washed with 3 L USP water and then with 1.9 L 1:1 ethanol SDA-2B:water. The filter cake was air dried for 4 hours and is then transferred to a vacuum oven. The solid was dried under vacuum at 45° C. until a constant mass was achieved, producing (S)-2-(5-(2-(5-ethyl-2-(4-methoxyphenyl)oxazol-4-yl)ethoxy)-2,3-dihydro-1H-inden-1-yl)acetic acid as an off white solid.

### Example 11

### Sodium (S)-2-(5-(2-(5-ethyl-2-(4-methoxyphenyl)oxazol-4-yl)ethoxy)-2,3-dihydro-1H-inden-1-yl)acetate

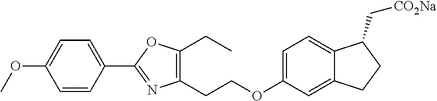

A 22 L reactor was charged with 3.8 L ethanol. Agitation was started, and the reactor was charged successively with 288.2 g sodium ethoxide solution (20.1% in ethanol) and with 378.4 g of (S)-2-(5-(2-(5-ethyl-2-(4-methoxyphenyl)oxazol-4-yl)ethoxy)-2,3-dihydro-1H-inden-1-yl)acetic acid (Example 10). The reaction mixture was heated to 40° C. for −20 minutes (until all solids are dissolved), and pH was checked (target pH 9-10).

The solution was filtered through a 10 micron filter membrane, returned to the reactor and heated to 40° C. The reactor was then charged with 3.4 L of filtered methyl t-butyl ether at such a rate that the temperature of the product solution is maintained at 40° C. throughout. The mixture is then seeded with 0.5 g Example 10 compound, and held at 42° C. for 40 minutes. An additional 3.4 L of filtered methyl t-butyl ether was added. The suspension was heated to 55° C. for 65 minutes. The suspension was cooled to 20-25° C. overnight then to 14° C. the next morning. The product was filtered under a nitrogen blanket, washed with 1.3 L filtered methyl t-butyl ether and dried to constant mass in a vacuum oven at 40° C. The bulk product was milled using a Comil with a 10 mesh sieve. The product is dried in a humidified environment at 40° C. NMR analysis showed ≤0.5% of ethanol by weight. Final product sodium (S)-2-(5-(2-(5-ethyl-2-(4-methoxyphenyl)oxazol-4-yl)ethoxy)-2,3-dihydro-1H-inden-1-yl)acetate was further dried at 45° C. under vacuum to obtain 306 g as a fine white solid.

### Example 12

### (S)-2-(5-(2-(2-(4-methoxyphenyl)-5-methyloxazol-4-yl)ethoxy)-2,3-dihydro-1H-inden-1-yl)acetic acid

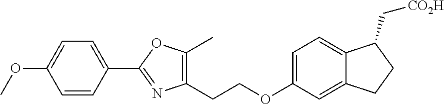 [SMILES: COc1ccc(-c2nc(CCOc3ccc4c(c3)CC[C@H]4CC(=O)O)c(C)o2)cc1]

(S)-Ethyl 2-(5-hydroxy-2,3-dihydro-1H-inden-1-yl)acetate from Example 1 and 2-(2-(4-methoxyphenyl)-5-methyloxazol-4-yl)ethanol from Example 3 were combined and reacted as in Examples 8, 9 and 10 to give (S)-2-(5-(2-(2-(4-methoxyphenyl)-5-methyloxazol-4-yl)ethoxy)-2,3-dihydro-1H-inden-1-yl)acetic acid as an off white solid.

### Example 13

### (S)-2-(5-(2-(5-ethyl-2-p-tolyloxazol-4-yl)ethoxy)-2,3-dihydro-1H-inden-1-yl)acetic acid

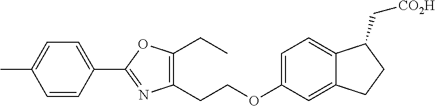 [SMILES: CCc1oc(-c2ccc(C)cc2)nc1CCOc1ccc2c(c1)CC[C@H]2CC(=O)O]

(S)-Ethyl 2-(5-hydroxy-2,3-dihydro-1H-inden-1-yl)acetate from Example 1 and 2-(5-ethyl-2-p-tolyloxazol-4-yl)ethanol from Example 4 were combined and reacted as in Examples 8, 9 and 10 to give (S)-2-(5-(2-(5-ethyl-2-p-tolyloxazol-4-yl)ethoxy)-2,3-dihydro-1H-inden-1-yl)acetic acid as an off white solid.

### Example 14

### (S)-2-(5-(2-(5-methyl-2-p-tolyloxazol-4-yl)ethoxy)-2,3-dihydro-1H-inden-1-yl)acetic acid

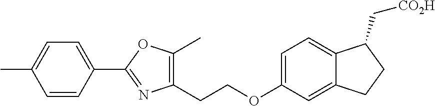 [SMILES: Cc1ccc(-c2nc(CCOc3ccc4c(c3)CC[C@H]4CC(=O)O)c(C)o2)cc1]

(S)-Ethyl 2-(5-hydroxy-2,3-dihydro-1H-inden-1-yl)acetate from Example 1 and 2-(5-methyl-2-p-tolyloxazol-4-yl)ethanol from Example 5 were combined and reacted as described in Examples 8, 9 and 10 to give (S)-2-(5-(2-(5-methyl-2-p-tolyloxazol-4-yl)ethoxy)-2,3-dihydro-1H-inden-1-yl)acetic acid as an off white solid.

### Example 15

### (S)-2-(5-(2-(5-ethyl-2-(4-ethylphenyl)oxazol-4-yl)ethoxy)-2,3-dihydro-1H-inden-1-yl)acetic acid

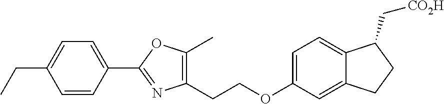 [SMILES: CCc1ccc(-c2nc(CCOc3ccc4c(c3)CC[C@H]4CC(=O)O)c(C)o2)cc1]

(S)-Ethyl 2-(5-hydroxy-2,3-dihydro-1H-inden-1-yl)acetate from Example 1 and 2-(5-ethyl-2-(4-ethylphenyl)oxazol-4-yl)ethanol from Example 6 were combined and reacted as described in Examples 8, 9 and 10 to give (S)-2-(5-(2-(5-ethyl-2-(4-ethylphenyl)oxazol-4-yl)ethoxy)-2,3-dihydro-1H-inden-1-yl)acetic acid as an off white solid.

### Example 16

### (S)-2-(5-(2-(5-ethyl-2-(4-ethylphenyl)oxazol-4-yl)ethoxy)-2,3-dihydro-1H-inden-1-yl)acetic acid

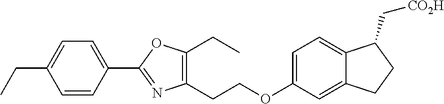 [SMILES: CCc1ccc(-c2nc(CCOc3ccc4c(c3)CC[C@H]4CC(=O)O)c(CC)o2)cc1]

(S)-Ethyl 2-(5-hydroxy-2,3-dihydro-1H-inden-1-yl)acetate from Example 1 and 2-(2-(4-ethylphenyl)-5-methyloxazol-4-yl)ethanol from Example 7 were combined and reacted as described in Examples 8, 9 and 10 to give (S)-2-(5-(2-(2-(4-ethylphenyl)-5-methyloxazol-4-yl)ethoxy)-2,3-dihydro-1H-inden-1-yl)acetic acid as an off white solid.

Demonstration of the activity of the compounds of the present invention may be accomplished through in vitro, ex vivo and in vivo assays that are well known in the art.

### Example 17

### NAFLD/NASH Animal Model

Twelve week old ZDF male rats (n=6 per group) were acclimated for 3 weeks prior to study initiation then fed with either a high fat (D12492+0.5% added cholesterol with 55% wt/vol fructose in drinking water) or low fat (DL12405J) diets. The compound of Example 10 above, vehicle, or pioglitizone were administered by oral gavage q.d. for 5 weeks as described in Table 1.

TABLE 1
Group Description and Dose Administration

| Group | Number of ZDF | | Dose Level | Dose |
|---|---|---|---|---|
| Number | Rats | Substance | (mg/kg) | Route |
| 1 | 6 | D12405J- | — | — |
| | | Low Fat | | |
| | | DietControl | | |
| 2 | 6 | Vehicle | — | PO |
| 3 | 6 | Example 10 | 0.3 | PO |
| 4 | 6 | Example 10 | 1.0 | PO |
| 5 | 6 | Example 10 | 3 | PO |
| 6 | 6 | pioglitizone | 3 | PO |

Body weights were recorded weekly, and blood samples were collected from all animals at the end of weeks 4, 5, 6 and 7 following an overnight fast, and triglycerides, cholesterol, AST, ALT, insulin, and BG were measured in singlet. After week 8, the livers were harvested from all animals and weighed. The left lobe was placed in 10% NBF for NASH scoring by PAI (H % E staining, Oil Red O staining and Sirius Red staining). One half of the right lobe was snap-frozen for qPCR (to quantify type 1 collagen, insulin receptor, IGF-1, ceramide synthase 2, sphingomyelin phosphodiesterase 3, MCP-1 and IL-6). The remaining right lobe was snap-frozen for estimation of liver triglycerides and cholesterol. The results from this study showed protection from NASH like symptoms in the high fat diet ZDF rats, for all three doses of compound of Example 10, in general similar to that provided by pioglitizone.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials and the like apparent to those skilled in the art still fall within the scope of the invention as claimed by the applicant.

What is claimed is:

1. A method of treating or preventing at least one of the following diseases:
a. Farber's Disease
b. ACLF (acute-on-chronic liver failure)
c. CLF (chronic liver failure)
d. POLT-HCV-SVR post-orthotopic liver transplant, or POLT, due to hepatitis C virus, or HCV, infection and have subsequently achieved sustained viral response, or SVR, following anti-HCV therapy
e. Alagille Syndrome
f. Progressive Familial Intrahepatic cholestasis (PFIC)
g. Primary Biliary Cirrhosis (PBC)
h. Primary Sclerosing Cholangitis
i. Autosomal Dominant Polycystic Liver Disease (ADPCLD)
j. Treatment of Liver Transplant Patients with reestablished fibrosis
k. Cholesteryl Ester Storage Disease (CESD)
l. Severe Hypertriglyceridemia (SHTG)
m. Homozygous Familial Hypercholesterolemia (HoFH)
n. Hepatic Encephalopathy (HE)
o. Nonalcoholic Steatohepatitis (NASH)
p. Nonalcoholic Fatty Liver Disease (NAFLD)
q. Alcoholic Liver Disease
comprising administering to a subject in need thereof an effective amount of a dual PPAR delta and gamma agonist.

2. A method according to claim 1 wherein the PPAR delta and gamma agonist comprises a compound of Formula I: 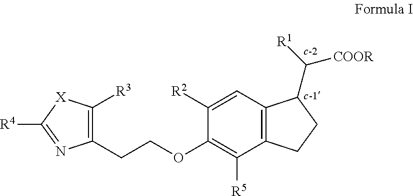
wherein in Formula I
R is H or C₁-C₆ alkyl;
R¹ is H, COOR, C₃-C₈ cycloalkyl, or C₁-C₆ alkyl, C₂-C₆ alkenyl, or C₁-C₆ alkoxy each of which may be unsubstituted or substituted with fluoro, methylenedioxyphenyl, or phenyl which may be unsubstituted or substituted with R⁶;
R² is H, halo, or C₁-C₆ alkyl which may be unsubstituted or substituted with C₁-C₆ alkoxy, oxo, fluoro, or
R² is phenyl, furyl, thienyl, pyrrolyl, oxazolyl, thiazolyl, imidazolyl, pyrazolyl, isoxazolyl, isothiazolyl, triazolyl, oxadiazolyl, thiadiazolyl, tetrazolyl, pyridyl, pyrrolidinyl, piperidinyl, tetrahydropyranyl, tetrahydrothiopyranyl, piperazinyl, or morpholinyl,
each of which may be unsubstituted or substituted with R⁶;
R³ is H, C₁-C₆ alkyl, or phenyl, which may be unsubstituted or substituted with R⁶;
X is O or S;
R⁴ is phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benxothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1,4-benzodioxanyl,
each of which may be unsubstituted or singularly or multiply substituted with R⁶, or with phenyl, furyl, thienyl, pyrrolyl, oxazolyl, thiazolyl, imidazolyl, pyrazolyl, isoxazolyl, isothiazolyl, triazolyl, oxadiazolyl, thiadiazolyl, tetrazolyl, pyridyl, pyrrolidinyl, piperidinyl, tetrahydropyranyl, tetrahydrothiopyranyl, piperazinyl, morpholinyl, benzodioxolyl, dihydrobenzofuranyl, indolyl, pyrimidinyl or phenoxy,
each of which may be unsubstituted or singularly or multiply substituted with R⁶;
R⁴ is C₁-C₆ alkyl or C₃-C₈ cycloalkyl, either of which may be unsubstituted or substituted with fluoro, oxo, or C₁-C₆ alkoxy which may be unsubstituted or substituted with C₁-C₆ alkoxy, or phenyl optionally substituted with R⁶,
each of which may be substituted with phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benzothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1,4-benzodioxanyl,
each of which may be unsubstituted or further substituted with R⁶, or
C₁-C₆ alkyl may also be substituted with C₃-C₈ cycloalkyl or with phenoxy which may be unsubstituted or substituted with R⁶ or with phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benxothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1,4-benzodioxanyl,
each of which may be unsubstituted or substituted with R⁶, or
R⁵ is H, halo or C₁-C₆ alkyl optionally substituted with oxo; and
R⁶ is halo, CF₃, C₁-C₆ alkyl optionally substituted with oxo or hydroxy, or
C₁-C₆ alkoxy optionally substituted with fluoro; or a pharmaceutically acceptable salt, ester prodrug, stereoisomer, diastereomer, enantiomer, racemate or a combination thereof.
R³ may be attached to the heterocyclic moiety of the compound of Formula I at either the 4 or 5 position (i.e., at either available carbon atom) and, accordingly, the remaining portion of the molecule will be attached at the remaining available carbon atom.

3. The method according to claim 1 wherein the PPAR delta and gamma agonist has the following structure: 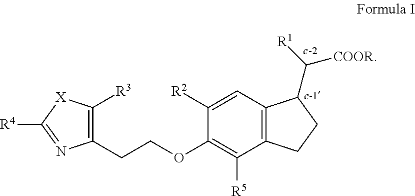
wherein in Formula I
R is H or C₁-C₆ alkyl;
R¹ is H;
R² is H, halo, or C₁-C₆ alkyl which may be unsubstituted or substituted with C₁-C₆ alkoxy, oxo, fluoro;
R³ is H, C₁-C₆ alkyl, or phenyl, which may be unsubstituted or substituted with R⁶;
X is O or S;
R⁴ is phenyl, which may be unsubstituted or singularly or multiply substituted with R⁶;
R⁵ is H, halo or C₁-C₆ alkyl optionally substituted with C₁-C₆ alkoxy, oxo, fluoro;
R⁶ is halo, CF₃, C₁-C₆ alkyl optionally substituted with oxo or hydroxy, or C₁-C₆ alkoxy optionally substituted with fluoro;
or a pharmaceutically acceptable salt, ester prodrug, stereoisomer, diastereomer, enantiomer, racemate or a combination thereof.
R³ may be attached to the heterocyclic moiety of the compound of Formula I at either the 4 or 5 position (i.e., at either available carbon atom) and, accordingly, the remaining portion of the molecule will be attached at the remaining available carbon atom.

4. The method according to claim 2 wherein: R is H or C₁-C₆ alkyl; R¹ is H; R² is H, halo; R³ is H, C₁-C₆ alkyl; X is O or S; R⁴ is phenyl, which may be singularly or multiply substituted with R⁶; R⁵ is H, halo; R⁶ is halo, CF₃, C₁-C₆ alkyl or C₁-C₆ alkoxy; and c-1′ has the S stereochemistry, or a pharmaceutically acceptable salt, ester prodrug, stereoisomer, diastereomer, enantiomer, racemate or a combination thereof.

5. The method according to claim 2 wherein: R is H; R¹ is H; R² is H, halo; R³ is C₁-C₆ alkyl; X is O; R⁴ is phenyl, which may be singularly or multiply substituted with R⁶; R⁵ is H, halo; R⁶ is halo, CF₃, C₁-C₆ alkyl or C₁-C₆ alkoxy; and c-1′ has the S stereochemistry or a pharmaceutically acceptable salt, ester prodrug, stereoisomer, diastereomer, enantiomer, racemate or a combination thereof.

6. The method according to claim 2 wherein: R is H, R¹ is H, R² is H, R⁵ is H, R³ is C₁-C₆ alkyl, X is S, and R⁴ is a phenyl, singularly or multiply substituted with R⁶, wherein R⁶ is halo, CF₃, C₁-C₆ alkoxyl or C₁-C₆ alkyl, and the stereochemistry at c-1′ is defined as S, or a pharmaceutically acceptable salt thereof.

7. The method according to claim 2 wherein: R is H, R¹ is H, R² is H, R⁵ is H, R³ is C₁-C₆ alkyl, X is O, and R⁴ is a phenyl, singularly or multiply substituted with R⁶, wherein R⁶ is halo, CF₃, C₁-C₆ alkoxyl or C₁-C₆ alkyl, and the stereochemistry at C-1′ is defined as S, or a pharmaceutically acceptable salt thereof.

8. The method according to claim 2 wherein: R is H, R¹ is H, R² is H or F, R⁵ is H or F, R³ is C₁-C₆ alkyl, X is O or S, and R⁴ is a phenyl, singularly or multiply substituted with R⁶, wherein R⁶ is halo, CF₃, C₁-C₆ alkoxyl or C₁-C₆ alkyl, and the stereochemistry at c-1′ is defined as S, or a pharmaceutically acceptable salt thereof.

9. The method according to claim 1 wherein the PPAR dual delta and gamma agonist has one of the following structures and is either the free acid or the potassium, sodium, calcium, magnesium, lysine, choline or meglumine salt: 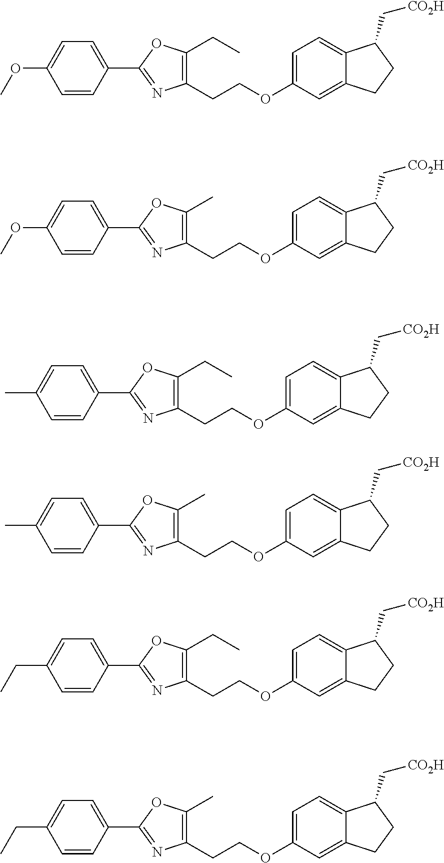 [SMILES: CCc1oc(-c2ccc(OC)cc2)nc1CCOc1ccc2c(c1)CC[C@H]2CC(=O)O ; COc1ccc(-c2nc(CCOc3ccc4c(c3)CC[C@H]4CC(=O)O)c(C)o2)cc1 ; CCc1oc(-c2ccc(C)cc2)nc1CCOc1ccc2c(c1)CC[C@H]2CC(=O)O ; Cc1ccc(-c2nc(CCOc3ccc4c(c3)CC[C@H]4CC(=O)O)c(C)o2)cc1 ; CCc1ccc(-c2nc(CCOc3ccc4c(c3)CC[C@H]4CC(=O)O)c(CC)o2)cc1 ; CCc1ccc(-c2nc(CCOc3ccc4c(c3)CC[C@H]4CC(=O)O)c(C)o2)cc1]
or a pharmaceutically acceptable salt, ester prodrug, or the potassium, sodium, calcium, magnesium, lysine, choline or meglumine salt thereof.

10. The method according to claim 9 wherein the PPAR dual delta and gamma agonist is a pharmaceutically acceptable salt thereof, wherein the pharmaceutically acceptable salt is selected from the group consisting of alkali metal salts, alkaline earth metal salts, ammonium salts with organic bases, and basic nitrogen containing groups in the conjugate base that is quaternized with agents selected from the group consisting of alkyl halides and aralkyl halides, or other alkylating agents.

11. The method according to claim 9 wherein the PPAR dual delta and gamma agonist is a potassium, sodium, calcium, magnesium, lysine, choline or meglumine salt thereof.

12. The method according to claim 2 wherein the PPAR dual delta and gamma agonist is administered intravenously, orally, buccally, transdermally, rectally, nasally, optically, intrathecally or intra-cranially.

13. The method according to claim 2 further comprising administration of one or more additional therapeutic agents.

14. The method according to claim 13 wherein one or more additional therapeutic agent is used to treat or prevent NASH disease or NAFLD.

15. The method according to claim 13 wherein one or more additional therapeutic agents is a farnesoid X receptor agonist such as obeticholic acid, aramchol, GR-MD-02, cysteamine bitartrate, simtuzumab, GFT-505, CER-002, KD3010, KD3020, MBX8025, metformin, rosiglitazone, pioglitazone, pentoxyfylline, vitamin E, selenium, omega-3 fatty acids and betaine.

16. The method according to claim 13 wherein the additional therapeutic agent is selected from the group consisting of anti-oxidants, anti-obesity agents, insulin sensitizers, anti-fibrotics, anti-dyslipidemics.

17. The method according to claim 13 wherein one or more additional therapeutic agents regulates hepatic steatosis, hepatic cirrhosis, or hepatic fibrosis.

18. The method according to claim 1 wherein the PPAR dual delta and gamma agonist is: (1S)-1H-Indene-1-acetic acid, 5-[2-[5-ethyl-2-(4-methoxyphenyl)-4-oxazolyl]ethoxy]-2,3-dihydro-, sodium salt (1:1).
